(19) United States
(12) Patent Application Publication
(10) Pub. No.: US 2009/0030723 A1
(43) Pub. Date: Jan. 29, 2009

(54) Method of genetic screening and analysis

(76) Inventor: Philip D. Buchanan, Chapel Hill, NC (US)

Correspondence Address: TRIANGLE PATENTS, P.L.L.C., P.O. BOX 28539, RALEIGH, NC, 27611-8539, US

(21) Appl. No.: 11/881,593

(22) Filed: Jul. 27, 2007

(51) Int. Cl. C40B 30/06; C12Q 1/68; G06Q 50/00

(52) U.S. Cl. 705/2; 435/6; 506/10

(57) ABSTRACT

A method of genetic screening and analysis, particularly for prenatal genetic screening to determine a risk estimate or probability of genetic disorders for a specific pregnancy integrating a patient's family history data (r1) with a patient's biological test data (r2p) to generate a final prenatal risk factor (Rf). A comprehensive risk factor (CRf) is generated by running a multiplicity of tests on the Rf for screening various genetic disorders.

### TECHNICAL FIELD

The present invention relates to methods of genetic screening and analysis. More particularly, the present invention relates to a method of prenatal screening that generates a final prenatal risk factor (Rf) for genetic defects by integrating a patient's family history data (r1) with a patient's biological test data (r2p). A comprehensive risk factor (CRf) is generated by running a multiplicity of tests on the Rf that screens for various genetic disorders.

### BACKGROUND OF THE INVENTION

Generally, current methods of prenatal genetic analysis include amniocentesis or chorionic villus sampling (CVS) and karyotyping. Amniocentesis is a procedure that extracts a small amount of amniotic fluid from the amnion around a developing fetus. The amniotic fluid houses a source of fetal cells that can be readily separated from the amniotic fluid for analysis. The chromosomes of the fetal cells are analyzed for genetic abnormalities. Typically, an amniocentesis is performed between 15 and 20 weeks gestation, however early amniocenteses can be performed as early as 13 weeks gestation. Due to the invasive nature of amniocentesis testing, there are potential risks involved including the introduction of pathogens into the amniotic sac from the needle, or the puncture wound to the amniotic sac not healing properly, leading to infections or leakage. Genetic amniocentesis is generally restricted to being performed no later than the second trimester, and is not performed early in pregnancies because there is not an ample source of amniotic fluid when the fetus is young.

CVS is another method commonly used for prenatal genetic analysis. CVS requires performing genetics tests on a sample of chorionic villus or placental tissue. A CVS procedure can be preformed as early as 10-12 weeks, however it has been found to result in increased risks of miscarriages. Both amniocentesis and CVS are invasive procedures involving risks to both the women and the fetus.

Recently, less invasive procedures have become available to screen for genetic defects that involve sampling a pregnant woman's blood, serum, or urine to screen for abnormalities in the levels of specific proteins or hormones that have been linked to specific birth defects. Fetal ultrasound exams in the first or second trimester can screen for anomalies or soft markers associated with certain genetic disorders, but such exams are costly and yield lower detections and higher false positive test results. One of the drawbacks of current non-invasive methods is that the screening is specific to three chromosome disorders. For example, the new nuchal translucency measures the fluid accumulation at the back of the fetal neck, which has been shown to be a strong marker for chromosomal abnormalities, including Down syndrome, Trisomy 18, and Trisomy 13. Additionally, two biochemical pregnancy protein markers in maternal blood freeBeta hCG and pregnancy associated plasma protein A (PAPP-A) have recently been shown to be highly specific and sensitive for Down syndrome and Trisomy 18 and 13. Additionally early detection methods include combining blood and precise ultrasound measurements to improve non-invasive screening.

Unlike CVS or amniocentesis, the present invention does not diagnose prenatal conditions, but instead provides a risk estimate or probability of genetic disorders specific to each pregnancy, thereby reducing the need for follow-up procedures such as CVS or amniocentesis. Thus, the present invention provides increased risk assessment accuracy by integrating a patient's age and family history data with biological test sampling to produce a final risk assessment (Rf), in which the Rf is subsequently screened for a multiplicity of genetic disorders.

The present invention is provided to solve the problems discussed above and other problems, and to provide advantages and aspects not provided by prior genetic screening methods of this type. A full discussion of the features and advantages of the present invention is deferred to the following detailed description, which proceeds with reference to the accompanying drawings.

### SUMMARY OF THE INVENTION

The present invention is directed to a method for genetic screening and analysis. The first aspect of the present invention is directed to a method of prenatal screening. The method comprises the steps of providing a first data set 10. The first data set 10 comprises patient data 12 of a patient. The patient data 12 is compared to a first standard 14 that provides a first risk factor (r1) 16 for genetic defects. The method further comprises providing a second data set 18. The second data set 18 comprises biological test data 20 from the patient. The biological test data 20 from the patient is compared to a second standard 22 that provides a second preliminary risk factor (r2p) 24 for genetic defects. The method further provides a final risk factor (Rf) 26 based upon an integration of r1 16 and r2p 24. Rf 26 provides increased accuracy prediction for genetic defects in a fetus of the patient.

In a second aspect of the claimed invention, the method of integrating r1 16 and r2p 24 is automated.

In another aspect of the claim invention a multiplicity of tests 28 are conducted on the Rf 26 to generate a comprehensive risk factor (CRf) 30 for determining risks for specific genetic defects.

Other features and advantages of the invention will be apparent from the following specification taken in conjunction with the following drawings.

### BRIEF DESCRIPTION OF THE DRAWINGS

To understand the present invention, it will now be described by way of example, with reference to the accompanying drawings in which:

FIG. 1 is a schematic drawing of the method of the present invention.

FIG. 2 is a graph of Down Syndrome Detection using risk factors of one embodiment of the present invention.

### DETAILED DESCRIPTION

While this invention is susceptible of embodiments in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

The present invention provides an improved method for screening prenatal genetic disorders by generating accurate and efficient risk assessments based on a multiplicity of factors. Furthermore, the present invention provides a method for a patient's fetus to be safely and accurately screened for potential risks of genetic disorders.

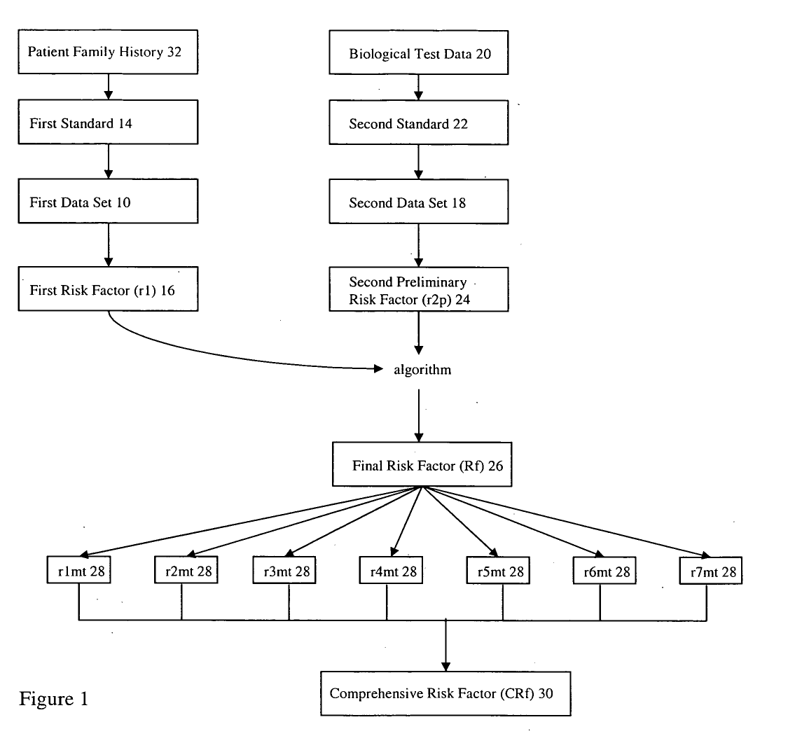

Referring now to the drawings in general, the illustrations are for the purpose of describing a preferred embodiment of the invention and are not intended to limit the invention thereto. As best seen in FIG. 1, a schematic shows an improved method for screening a patient's fetus for genetic disorders by integrating a first risk factor (r1) 16 with a second preliminary risk factor (r2p) 24 to generate a final risk factor (Rf) 26. The schematic of FIG. 1 provides a first data set 10. The first data set 10 comprises patient data 12 in the form of a patient's family history 32. A patient's family history may include a patient's background information, including but not limited to the following: date of birth (DOB), egg donor age, egg donor date of birth, weight, ethnicity, last menstrual period (LMP), gestational age by ultrasound dating, gestational age by LMP, gestational age by in vitro fertilization (IVF), twins, zygosity, multiple pregnancies (3, 4, etc.) crown rump length (CRL), nuchal translucency measurements in mm (NT), present or absent nasal bone (NB), tricuspid valve flow (TF), frontal maxillary facial angle (FMFA), family history (FH) of Down syndrome (DS), family history of trisomy 18, family history of open spine or skull defect, carbamazepine or valproic acid medication, and insulin dependent diabetes medication (IDDM). When gather the patient's information, the family history data 32 may be selected from a series of predetermined family codes 36 generated by software programs known in the art to assist a technician with data entry. In one embodiment, a patient's family history 32 may already be stored in an existing database based on a previous visit, for example testing results from a previous pregnancy or repeat maternal serum alpha-fetoprotein (MSAFP) detection results. In such an event, there is no need for the patient to re-submit the information previously stored in the database, other than to update the patient's family history with any new or additional information. Consequently, method of the present invention reduces the amount of time required by the technician to enter family history data and ensures that a patient's family history is accurately updated. Unlike large general laboratories, the present invention does not use default data such as weight or ethnicity default data. For example, if patient information such as weight or family history is not available, the present invention obtains current data from the referring physician prior to analysis.

The first data set 10 is compared to a first standard 14 that generates a first risk factor (r1) 16. In the preferred embodiment, the first standard 14 is the family history of a 35-year-old patient with no history of genetic defects. This biological standard is used to select patients as within normal range or at increased risk because their risk is lower or higher than the 35-year-old standard cutoff. Test results are calculated as a risk ratio, e.g. 1/100, with the same standard applying to all patients. The first risk factor (r1) 16 provides a risk assessment based only on a patient's family history and current physiological conditions.

As shown in the schematic of FIG. 1, a second data set 18 is provided. The second data set 18 comprises biological test data 20 from a patient. The biological test data 20 is generated by obtaining a biological sample from the patient such as, but not limited to, blood, serum or urine samples. In one embodiment of the present invention dried blood samples are used because dried blood stabilizes the blood proteins and eliminates sample degradation, thus ensuring that the patient's sample is representative of her true biological state. The patient's biological sample is screened for genetic defects using known methods. For example, the results of biological testing for freeBeta and PAPP-A on dried blood specimens are compared to test data for normal and affected patients. Biological test data is also adjusted for the presence of patient factors that affect the protein level in patient's blood sample such as, but not limited to ethnicity. The standards for biological testing are also selected based on the observed levels of proteins in affected and unaffected patients and the assay coefficient of variance. The test results of the biological sample are then compared to a second standard 22 to generate a second preliminary risk factor (r2p) 24 similar to the first standard 14 discussed above. In the preferred embodiment, the second standard 22 is the biological test results of a 35-year-old patient with no history of genetic defects.

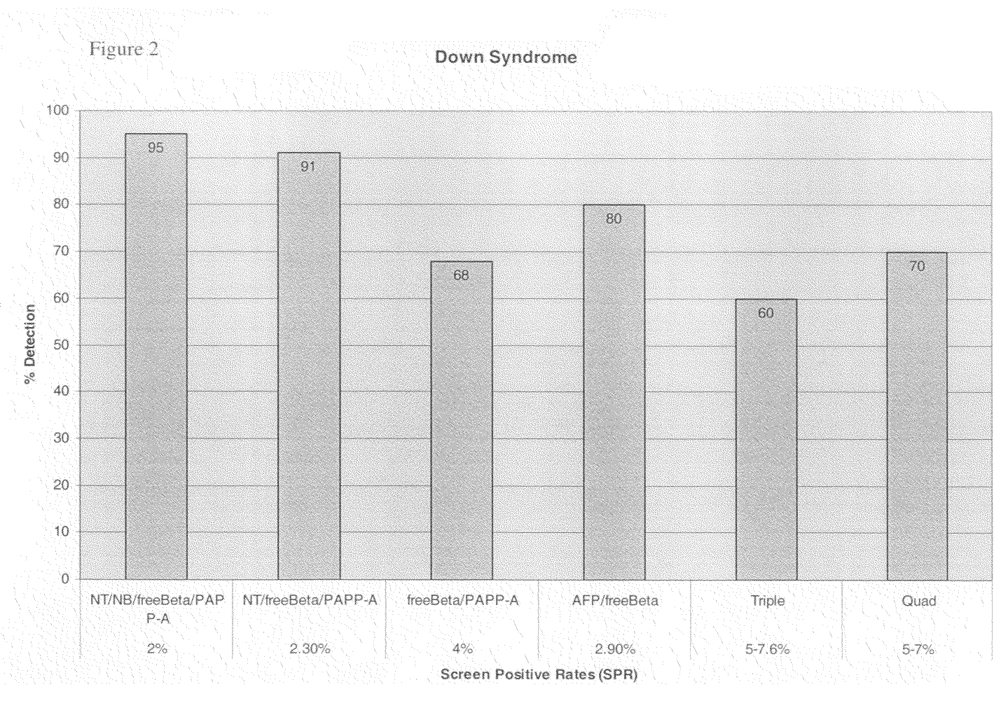

As further shown in FIG. 1, a final risk factor (Rf) 26 is generated by a weighted integration of r1 16, patient and/or family history; and r2p 24, biological test results and ultrasound measurement risks. The r1 16 is combined with the r2p 24 using an algorithm, and the combined risk is compared to the normal and affected control data to generate a final risk factor (Rf) 26 for Down syndrome, trisomy 13/18 chromosome abnormalities, and other specific risks for a genetic disorder to a specific pregnancy. Each patient's risk is designated as within normal range or at increased risk. Depending on the possible ratios of each of the elements being factored, the weight of each factor being considered is increased or decreased to provide a higher weight for the more effective risk factors. As shown in FIG. 2, NT/NB/freeBeta/PAPP-A and AFP/freeBeta are the most sensitive and specific when screening for Down syndrome. Significantly, the present invention provides a unique method of integrating patient data in the form of family history with biological tests of a patient to generate a final risk factor (Rf) 26. In one embodiment the integration of r1 and r2p is automated, using an algorithmic software to combine r1 and r2p to yield optimal detection. The Rf 26 provides more accurate and reliable risk assessments of potential genetic disorders.

One of the significant advantages of the method of the present invention is that testing is not restricted to a specific week during a patient's pregnancy. Since the method of the present invention is non-invasive, genetic screening can be conducted during an optimal week range appropriate for testing a specific risk factor, without putting the patient or fetus in danger. In a preferred embodiment, biological testing for freeBeta and PAPP-A are most effective at between about 24 and about 84 mm CRL, at 9 weeks, 1 day-13 weeks, 6 days. In another embodiment of the present invention, an automatic stop of the screening is triggered if the pregnancy is outside the appropriate week range to prevent human error and miscalculations of risk factors. For example, screening may be considered too early if a fetus' CRL is less than 24 mm. Alternatively, screening may be considered too late if a fetus' CRL is more than 84 mm.

In one embodiment of the present invention, a multiplicity of tests 28 is conducted on the Rf 26 to generate a comprehensive risk factor (CRf) 30 with increased accuracy for genetic detections. The multiplicity of tests 28 comprises a series of individual tests 34, r1mt, r2mt, r3mt, r4mt, r5mt, r6mt, r7mt etc., which screen for a specific genetic defect. The multiplicity of tests includes, but is not limited to, testing for Down syndrome, Trisomy 18/13, nasal bone defects, tricuspid flow defects, frontal maxillary angle defects, nuchal translucency, singletons, and monozygotic or dizygotic twins. Significantly, the method of the present invention allows simultaneous screening for multiple genetic disorders on the Rf 26. The screening method of the present invention provides patient's-specific risk for Trisomy 13 and Trisomy 18 rather than Trisomy 18 alone, detects about 40% of fetal heart defects, detects other types of fetal anomalies, and provides a risk estimation for some perinatal factors such as, but not limited to, pre-term delivery. Significantly, since the Rf 26 can be screened for numerous genetic abnormalities at once, without conducting separate, individual tests for each abnormality being screened, the present invention is more efficient and cost effective than previous prenatal genetic screening methods.

### EXAMPLES

The following Table is a comparison of prenatal screening programs, including detection rates and screen positive rates for determining the weight of each risk factor. More weight is given for the more effective risk factors. Combining ultrasound measurements for Nuchal Translucency, dried blood freeBeta, and PAPP-A provide higher detection than older methods of screening for Down syndrome and Trisomy 18. Nasal bone or Tricuspid Flow can be added to increase detection by 4-5%.

| | First Trimester Detection Rates & Screen Positive Rates | | | | |
|---|---|---|---|---|---|
| | | Early | | Research/Experimental | |
| | | Screen ® | | Non-FMF | |
| | | With | | NT/hCG/PAPP- | Integrated |
| | Early Screen ® | Nasal | | A (No USA | and |
| | FMF/NT/freeBeta/PAPP-A | Bone | FreeBeta/PPAP-A | Ref. | Sequential* |
| Down Syndrome (DS) | 91% at 2.3% | 95% at | 68% at 4.5% | 82% at 5-7% | Varies* |
| | | 2% | | | (82-86%) |
| Trisomy 18 | 97% at 0.4% | 98% at | >90% at 0.4% | No USA Ref. | No USA |
| | | 0.4% | | | Ref. |
| Trisomy 13 | 97% at 0.1% | 98% at | 90% at 0.1% | No | No |
| | | 0.1% | | | |
| Twins | 80% at 7.2% | 80% at | 50% | No USA Ref. | No |
| | | 7.2% | | | |
| Heart Defeats/Anomalies | 40%/Yes | 40%/ | No/Yes | No | No USA |
| | | Yes | | | Ref. |
| Diabetics/Multiples/T-18/ | Yes | Yes | No | No | No |
| T-13/Nasal Bone | | | | | |
| Dried Blood freeBeta | Yes | Yes | Yes | No | No |
| FMF NT Compatible Lab | Yes | Yes | Yes | No | No |
| Screening Range (weeks) | 11-13⁶ w | 11-13⁶ w | 9-13⁶ w | 10-13⁶ w | 18-20 w |
| FMF Database | Yes | Yes | Yes | No | No |

While the specific embodiments have been illustrated and described, numerous modifications come to mind without significantly departing from the spirit of the invention, and the scope of protection is only limited by the scope of the accompanying Claims.

What is claimed is:

1. A method for prenatal screening, comprising the steps of:
a) providing a first data set, wherein the first data set comprises patient data of a patient that compared to a first standard provides a first risk factor (r1) for genetic defects;
b) providing a second data set, wherein the second data set comprises biological test data from the patient that compared to a second standard provides a second preliminary risk factor (r2p) for genetic defects; and
c) generating a final risk factor (Rf) based upon an integration of r1 and r2p, wherein the Rf provides increased accuracy of prediction for genetic defects in a fetus of the patient.

2. The method of claim 1, wherein the patient data comprises a patient's family history comprising date of birth (DOB), egg donor age, egg donor date of birth, weight, ethnicity, last menstrual period (LMP), gestational age by ultrasound dating, gestational age by LMP, gestational age by in vitro fertilization (IVF), twins, zygosity, multiple pregnancies (3, 4, etc.) crown rump length (CRL), nuchal translucency measurements in mm (NT), present or absent nasal bone (NB), tricuspid valve flow (TF), frontal maxillary facial angle (FMFA), family history (FH) of Down syndrome (DS), family history of trisomy 18, family history of open spine or skull defect, carbamazepine or valproic acid medication, or insulin dependent diabetes medication (IDDM).

3. The method of claim 2, wherein the patient family history may be selected from a series of predetermined family codes.

4. The method of claim 1, wherein the first standard is a 35-year-old patient with no history of genetic defects.

5. The method of claim 1, wherein the biological test data comprises blood tests, free Beta tests or PAPP-A tests.

6. The method of claim 1, wherein the final risk factor is generated by a weighted integration of r1 and r2.

7. The method of claim 1, wherein the screening is conducted during a week range appropriate for testing specific risk factors.

8. The method of claim 1, wherein the screening of a fetus with a CRL of 24-82 mm is conducted during a week range from about 9 weeks and 1 day to about 13 weeks and 6 days.

9. The method of claim 7, wherein the screening automatically stops if a pregnancy is outside the appropriate week range to prevent human error.

10. A method for prenatal screening, comprising the steps of:
a) providing a first data set, wherein the first data set comprises patient data of a patient that compared to a first standard provides a first risk factor (r1) for genetic defects;
b) providing a second data set, wherein the second data set comprises biological test data from the patient that compared to a second standard provides a second preliminary risk factor (r2p) for genetic defects;
c) generating a final risk factor (Rf) based upon an integration of r1 and r2p, wherein the Rf provides increased accuracy of prediction for genetic defects in a fetus of the patient; and
d) running a multiplicity of tests on the Rf to generate a comprehensive risk factor (CRf) for determining risks for specific genetic defects.

11. The method of claim 10, wherein the multiplicity of tests comprises a series of individual tests that screen for a specific genetic defect.

12. The method of claim 11, wherein the individual test includes testing for Down syndrome.

13. The method of claim 11, wherein the individual test includes testing for nasal bone defects.

14. The method of claim 11, wherein the individual test includes testing for Trisomy 18.

15. The method of claim 11, wherein the individual test includes testing for Trisomy 13.

16. The method of claim 11, wherein the individual test includes testing for tricuspid flow defects.

17. The method of claim 11, wherein the individual test includes testing for frontal maxillary angle defects.

18. The method of claim 11, wherein the individual test includes testing for nuchal translucency.

19. The method of claim 11, wherein the individual test includes testing for singletons.

20. The method of claim 11, wherein the individual test includes testing for monozygotic or dizygotic twins.

21. The method of claim 10, wherein the integration of r1 and r2p is automated.

22. The method of claim 10, wherein the multiplicity of steps are conducted simultaneously on the Rf when generating the CRf.
